(19) United States
(12) Patent Application Publication
(10) Pub. No.: US 2014/0023583 A1
(43) Pub. Date: Jan. 23, 2014

(54) METHOD OF MEASURING AND COMPARING LEVELS OF SUBSTANCES IN AN INDIVIDUAL'S BODY

(71) Applicant: William J. McFaul, (US)

(72) Inventor: William J. McFaul, Jackson, NJ (US)

(21) Appl. No.: 14/036,147

(22) Filed: Sep. 25, 2013

Related U.S. Application Data
(63) Continuation of application No. 12/701,083, filed on Feb. 5, 2010.
(60) Provisional application No. 61/150,478, filed on Feb. 6, 2009.

(51) Int. Cl. A61K 51/00

(52) U.S. Cl. CPC A61K 51/00
USPC 424/1.11

(57) ABSTRACT

A method for measuring the homeostatic relationship between various substances in the body. The measured levels of substances in the body use the interrelationships of the various substances in order to establish guidelines for treating individuals.

This patent application is a continuation of U.S. application Ser. No. 12/701,083, filed Feb. 5, 2010, which claims the benefit of U.S. Provisional Application Ser. No. 61/150,478, filed Feb. 6, 2009, the contents of each of which are incorporated herein by reference in their entirety.

### BACKGROUND OF THE INVENTION

1. Field of the Invention

The present invention relates to the field of diagnosing the levels of substances in the body of individuals. In particular the present invention illustrates the measuring of levels of neurohormones and neurotransmitters, minerals, elements, vitamins, genes, proteins, peptides, enzymes, hormones, lipids (i.e. fatty acids), carbohydrates, amino acids, vitamers, ions and gasotransmitters in an individual for purposes of diagnosis.

2. Description of the Related Technology

All forms of life are supported by a variety of substances; including but not limited to hormones, proteins, peptides, amino acids, minerals (ions), vitamins (chemicals or compounds that function as vitamins) and bacteria. These substances create the homeostasis essential to maintain life for humans as well as plants and animals.

Current scientific findings have not identified that various pairs of substances affect levels of each other in the body. Furthermore, certain other substances modulate (regulate) levels of the pairs of substances. Additionally, certain substances are a part of more than one modulated pair of substances. These relationships can be utilized in order to determine various health parameters of an individual, however, to date; these corollary relationships have not been diagnosed in order to establish treatments for individuals.

There are currently accepted, statistically valid ranges for safe levels of substances in the body. These levels are established by science and used widely in diagnosis, medical testing and research today. Substances can include, but not be limited to: minerals, elements, vitamins, genes, proteins, peptides, enzymes, hormones, lipids (i.e. fatty acids), carbohydrates, amino acids, vitamers, ions and gastro-transmitters. These levels are both detected and have been established as part of past and current research that has included and includes various diagnostic detection and testing methodologies in use in the marketplace. Depending upon the substances and combination thereof, many different tests and diagnostic analyses exist to test for different substances. Some of these tests are predictive (showing indicators of potential problems; i.e. illness or disease), and some are used to identify the existence or presence of illness or disease. In most cases, the configuration of the substances determines the test. These substances are almost always those which medical and scientific research has identified as being related to illness or disease states. In some cases, they may be substances which are being investigated for potential relation to illness or disease. These modulated pairs and their relationships offer the ability to create and perform multiple tests for use in both the marketplace and private research.

Therefore, there is a need in the field to identify and utilize the homeostatic relationship between the substances that are important to the body's functioning via the measuring of various levels of the substances that are part of these pairings.

### SUMMARY OF THE INVENTION

An object of the invention is the establishing a diagnosis based upon detectable substances in the body including minerals, elements, vitamins, genes, proteins, peptides, enzymes, hormones, lipids (i.e. fatty acids), carbohydrates, amino acids, vitamers, ions, gasotransmitters and neurotransmitters.

Another object of the invention is the measuring of levels of detectable substances in the body including minerals, elements, vitamins, genes, proteins, peptides, enzymes, hormones, lipids (i.e. fatty acids), carbohydrates, amino acids, vitamers, ions, gasotransmitters and neurotransmitters.

An aspect of the present invention may be a method of determining a diagnosis comprising; measuring a level of a first substance in an individual; measuring a level of a second substance in an individual; measuring a level of a third substance in an individual; comparing the measured level of the first substance to the measured level of the second substance; comparing the measured level of the second substance to the measured level of the third substance and comparing the measured level of the third substance to the measured level of the first substance; and wherein the compared measured levels are further compared to predetermined levels to establish a diagnosis.

Another aspect of the present invention is a method for establishing a diagnosis based upon compared levels of neurohormones: measuring a level of aldosteione in an individual; measuring a level of oxytocin in the individual; measuring a level of serotonin in the individual; measuring a level of adrenaline in the individual; measuring a level of cortisol in the individual; comparing the measured levels; and establishing a diagnosis based upon the compared measured levels.

Still yet another aspect of the present invention is a method for establishing a diagnosis based upon compared levels of amino acid neurotransmitters: measuring a level of glutamate in an individual; measuring a level of glycine in the individual; measuring a level of cysteine in the individual; measuring a level of aspartate in the individual; measuring a level of gamma aminobutyric in the individual; comparing the measured levels; and establishing a diagnosis based upon the compared measured levels.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 illustrates the relationship of a modulated pair.

FIG. 2 shows the relationship between aldosterone, oxytocin, serotonin, adrenaline and cortisol.

FIG. 3 shows the method of measuring levels within an individual using aldosterone, oxytocin, serotonin, adrenaline and cortisol, in accordance with an embodiment of the present invention.

FIG. 4 shows the relationship between glutamate, glycine, cysteine, aspartate and gamma aminobutyric acid.

FIG. 5 shows the method of measuring levels within an individual using glutamate, glycine, cysteine, aspartate and gamma aminobutyric acid, in accordance with an embodiment of the present invention.

FIG. 6 shows the relationship between sodium, potassium and chloride.

FIG. 7 shows the method for measuring levels within an individual using sodium, potassium and chloride.

### DETAILED DESCRIPTION OF THE PREFERRED EMBODIMENT(S)

All foams of life are supported by modulated pairs of substances; including but not limited to minerals, elements, vitamins, genes, proteins, peptides, enzymes, hormones, lipids (i.e. fatty acids), carbohydrates, amino acids, vitamers, ions, gastro-transmitters and neurotransmitters. These substances create the homeostasis essential to maintain life for humans as well as plants and animals.

Several terms are used herein and are defined as follows:

The term “modulator” means an agent, substance or mechanism that facilitates the maintenance of homeostasis between pairs. “Pairs” may be hormones, minerals, proteins, amino acids, bacteria, virus, and natural processes. The agent can decrease intensity of stimulatory processes or substances, or increase intensity of compensatory ones.

The term “catalyst” means the driver which initiates modulation to take place. Such catalysts can include enzymes, hormones, RNA, and countless signaling mechanisms.

The term “neuron” means the signaling molecules or signaling mechanisms.

The term “substance” refers to compounds, elements, ions and molecules and combinations thereof.

The term “Homeostatic Relationship (Homeostasis)” means the balance or equilibrium between two substances. The relationship does not have to be 50%-50% but can vary depending upon the two substances in question.

The invention addresses the diagnostic analysis of the corollary relationships between substances that provide and maintain homeostasis; i.e. pairs which include substances that are stimulatory/excitatory/positively charged and those that are compensatory/inhibitory/negatively charged as well as substances that perform modulatory functions to support or maintain the homeostatic relationship between substances.

The data for measuring homeostasis between substances within a modulated pair can vary from one individual or specimen to another due to environmental factors, basic metabolism, genetic make-up and other factors. Accordingly, a range of homeostatic relationships will need to be determined for all modulated pairs in order to assess deviations and disruptions.

By utilizing the analysis of data relative to the ranges of homeostasis and the deviations thereof, medical science (for humans as well as well as in veterinary medicine for animals) will be able to manufacture new pharmaceutical products and other substances, prepare diets, manufacture devices, create therapies, compound personalized nutrients or substances of various types in order to maintain or regulate homeostasis.

Data derived from the application of the invention will allow for diagnostic analysis and control of levels of substances in order to prevent their depletion, to maintain adequate levels to prevent disruption as well as to create recommendations for which substances may counteract with others and the levels of which substances must be modified on an ongoing basis in order to provide optimal health/well being through homeostasis.

Since nutrients, such as vitamins, minerals, electrolytes, enzymes, amino acids, proteins, etc, are essential to create defenses for the organs, glands and operating systems of the body, as well as to provide the energy to sustain life, establishing corollary relationships between substances that constitute “nutrients” will enable cross-category analysis to prevent depletion of the body's defense in order to prevent illness as well as to increase levels of nutritional energy to strengthen the body to offset the effects of existing ailments and diseases.

Data derived though the application of the invention will enable scientists to monitor the impacts of various chemicals, natural products; including herbs and foods and medications as they can impact hormones, chromosomes, RNA, genes/DNA, rates of cellular absorption and the firing rates of neurons/signaling mechanisms. An example of such impacts on the body is found in scientific studies that identify the ability of numerous chemicals to disrupt hormones and/or genes.

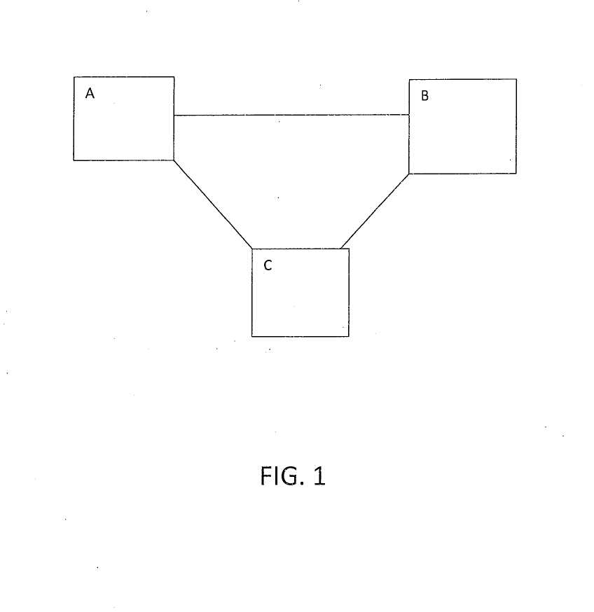

The concept of modulation in relation to homeostatic pairs is illustrated in FIG. 1. This example illustrates a relationship between two substances, A and B, and the existence of a third substance C that serves to support A or B in order to attempt to maintain an adequate level of homeostasis, i.e. balance, between the two substances. The model of a modulated pair remains a constant; there are 3 substances which have a mutual relationship consisting of a “base pair” “A” and “B” which is modulated by “C.” The base pair of substances, A and B, have a polarizing/mutualistic relationship that has a direct effect on the “level” (detectable amount) of each. The modulator, C, has a direct effect on either A or B in that it can lower or raise the level of either (and subsequently the pair), depending upon the amount of modulator present. For example, high levels of one substance in relation to another in a base pair will lower the other substances level in the same test subject.

The base pair levels have a natural homeostatic balance. Each component of the base pair has a level that can be determined individually, i.e. A and B separately, and in relation to each other, i.e. A in ratio to B. The individual's normal/safe levels for each substance are based on current values established by medical and scientific research and are standards used today. This homeostatic value is established by evaluating a ratio. These pairs can become disproportionately imbalanced; i.e. pair members (either A or B and or A in relationship to B) can reach unsafe levels. The modulator C can either cause the imbalance, i.e. if there is an excessive amount of the substance present and determined by identifying the level or help to restore balance (again, depending upon the level). By identifying these imbalances detection and diagnosis of illnesses and diseases of the body may be made. Depending upon the modulated pair being analyzed, chronic imbalances may be the sign of more serious/impending illness or disease potential. Sporadic imbalances may be a predictive sign of illness or disease potential.

Some additional examples of modulated pairs in science and in the body include calcium and magnesium modulated by fluorine. Other examples include, aldosterone and oxytocin modulated by serotonin; adrenaline and cortisol modulated by oxytocin; norepinephrine and prolactin modulated by dopamine; glucose and insulin modulated by glucagon; red blood cells and white blood cells modulated by platelets; phosphatidylcholine (PC) and phosphatidylserine (PS) modulated by dimethylaminoethanol (DMAE). The pair of PC and PS contains acetylcholine which is the primary neurotransmitter in the central nervous system.

Further examples of modulated pairs are as follows: sodium and potassium modulated by chloride having the role/process of signaling for skeletal muscles; manganese and cobalt modulated by iron having the role/process of signaling for amyloid and for one three bacterial immune processes; copper and zinc modulated by calcium and manganese having the role/process of signaling for one of the three bacterial immune processes; molybdenum and copper modulated by sulfur having the role/process of signaling for one of the three bacterial immune processes; selenium and zinc modulated by iodine having the role/process of signaling for one of the three viral immune processes; aluminum and iron modulated by phosphorus having the role/process of signaling for one of the three viral immune processes; sulfur and iron modulated by zinc having the role/process of signaling for one of the three viral immune processes; copper and zinc modulated by silver having the role/process of signaling for one of the three mold immune processes; nickel and titanium modulated by manganese having the role/process of signaling for one of the three mold immune processes; molybdenum and copper modulated by sulfur having the role/process of signaling for one of the three mold immune processes; vanadium and chromium modulated by phosphorus having the role/process of signaling for muscle activity; calcium and phosphorus modulated by magnesium having the role/process of signaling for parathyroid activity; calcium and phosphorus modulated by magnesium and vitamin D3 having the role/process of signaling for bone synthesis; calcium and magnesium modulated by fluorine having the role/process of signaling for intra-cellular energy; molybdenum and tungsten modulated by vanadium having the role/process of signaling for blood glucose process; lithium and sodium modulated by calcium having the role/process of signaling between adrenaline and cortisol; nickel and zinc modulated by boron having the role/process of signaling for smooth muscle tissue; bromine and iodine modulated by chlorine and fluorine having the role/process of signaling for digestive enzyme activity; copper and zinc modulated by magnesium having the role/process of signaling for emotion neurohormone activity; Tumor Necrosis Factor A and Tumor Necrosis Factor B modulated by Interleukin 10 having the role/process of signaling for apoptosis of mutated cells; carbon monoxide and carbon dioxide modulated by nitric oxide having the role/process of signaling for homeostasis of gasto-transmitters; iron and molybdenum modulated by sulfur having the role/process of signaling for nitrogenase; Dihomo gamma linolenic Acid (DGLA) and Arachidonic acid (AA) modulated by gamma Linolenic acid (GLA) having the role/process of components for formation of eicosanoids; Dihomo gamma Linolenic acid (DGLA) and Eicosapentaenoic acid (EPA) modulated by Erucic Acid having the role/process of affecting lipids for smooth muscle constriction; Eicosapentaenoic acid (EPA) and Docosahexaenoic acid (DHA) modulated by Clupanodonic acid and Osbond Acid (DPA) having the role/process of affecting lipids for artery and blood vessel maintenance; Very-low density lipoprotein (VLDL) and Chylomicrons modulated by Intermediate density lipoprotein (IDL) having the role/process of being a component of lipid profiling panel; Low density lipoprotein (LDL) and High density lipoprotein (HDL) modulated by Lipoprotein (a) (LDA) having a role/process of being components to synthesize cholesterol; Triiodothyronine (T3) and Thyroxine (T4) modulated by Diiodothyronine (T2) having the role/process of being components of thyroid hormone; Protein Gli 1 and Protein Gli 3 modulated by Protein Gli 2 having the role/process of providing signals for tissue inflammation; smoothened protein (SMO) and Cubitus interruptus protein (Ci) modulated by Costal 2 having the role/process signaling tissue growth; Cephalin and Sphingomyelin modulated by Phosphatidylserine (PTD-L-SER) having the role/process of being Lipids essential for synthesis of myelin; Resistin and Chemerin modulated by Plasminogen activator inhibitor having the role/process of signaling for Adipose Derived Hormones; Heat Shock Protein 60 (HSP 60) and Heat Shock Protein 100 (HSP 100) modulated by Heat Shock Protein 70 (HSP 70) having the role/process of being dynamics for foiming amino acid links to created proteins, peptides and hormones; Heat Shock Factor 1 (HSF1) and Heat Shock Factor 2 (HSF2) modulated by Nucleoporin p62 having the role/process of signaling for Heat Shock Proteins; Phosphatidylcholine (PC) and Phosphatidylserine (PS) modulated by Dimethylaminoethanol (DMAE) having the role/process of being components of acetylcholine (the CNS primary neurotransmitter); C. Difficile Toxin A and C. Difficile Toxin B modulated by C. Difficile Binary Toxins having the role/process of modulating the homeostasis of C. difficile bacteria; Phylloquinone (K1) and Menaquinone (K2) modulated by Menaquinone (MK4) having the role/process of providing synthesis of vitamin K; Phosphatidylinositol 3 and phosphate (PI[3]P) Phosphatidylinositol 4—phosphate (PI[4]P) modulated by Phosphatidylinositol 5—phosphate (PI[5]P) having the role/process of being Phospholipids for foundation of cell formation; Phosphatidylinositol 3,4 bisphosphate (PI[3,4]P2) Phosphatidylinositol 3,5—bisphosphate (PI[3,5]P2) modulated by Phosphatidylinositol 4,5—bisphosphate (PI[4,5]P2) having the role/process of being Phospholipids for foundation of cell formation; Afferent Neurons and Efferent Neurons modulated by Interneurons having the role/process of pairing of neurons; Glucose and Insulin modulated by Glucagon having the role/process of affecting the blood sugar process; Estradiol (E3) and Estriol (E2) modulated by Estrone (E1) having the role/process of being part of the Estrogen composition; Progesterone (PROG) and Dehydroepiandrosterone (DHEA) modulated by Pregnenolone (PREG) having the role/process of pairing Neurosteroids; Protein HO-2 and Protein HO-3 modulated by Protein HO-1 having the role/process of being enzymes for lung activities; Pyridoxal and Pyridoxamine modulated Pyridoxine having the role/process of being components that form the enzyme Glutamate decarboxylase (GAD); Hedgehog (Hh) and PTCH-1 modulated by the smoothened protein (SMO) having the role/purpose of providing homeostasis for process of cell growth; Copper and Zinc modulated by Cyanide having the role/purpose of providing components of the copper-zinc superoxide enzyme; Oleuropein and Oleocanthal modulated by Hydroxytyrosol having the role/process of being components of the brain's anti-microbial defenses; Norepinephrine and Prolactin modulated by Dopamine having the role/purpose of being neurohormones for logic and decision making; Adrenaline and Cortisol modulated by Oxytocin having the role/purpose of being Neurohormones for emotions; Aldosterone and Oxytocin modulated by Serotonin having the role/purpose of being Neurohormones for emotions; Gamma Aminobutyric Acid and Aspartate modulated by Glutamate having the role/process of regulating the neural firing rate. It should be noted that the role/process indicated for each “pair” is merely for illustrative purposes, in some instances, these “pairs” of substances may perform multiple roles in the body depending upon location; e.g. in the brain as opposed to in the body's extra-cellular matter.

Some substances can function as part of a pair and also be a modulator for another pair. For example, noted above was the pair of aldosterone and oxytocin which is modulated by serotonin. Oxytocin also operates as a modulator for the pair of adrenaline and cortisol. Another example of this can be the pair of glutamate and glycine modulated by cysteine Glutamate can also operate as a modulator of the base pair of aspartate and gamma amino butyric acid.

Correlation between base values expressed when the range of homeostasis is determined and individual test results can be used as a means of assessing levels or disruptions between pairs or the existence of increases in the levels of modulators in order to maintain homeostasis. Methods of diagnostic analysis may also detect the presence of catalysts as markers for the existence of a disruption within a modulated pair that the body is in the process of correcting. Detection of such catalysts may be included as a factor in the process of analyzing the correlation of substances within modulated pairs.

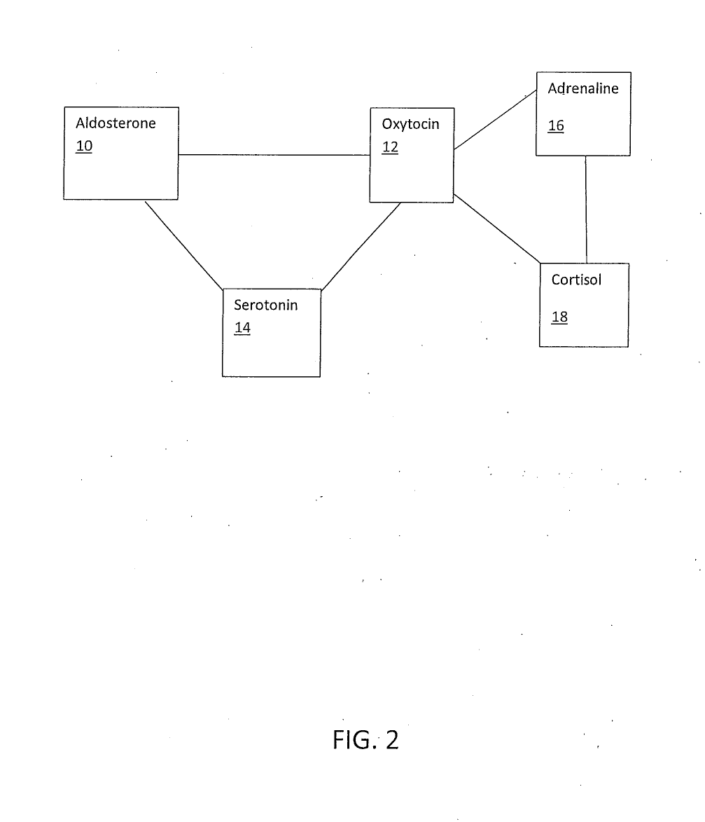

Described herein is an example of the determination of homeostatic levels of two of the sets of modulated pairs discussed above. The first set is the pair of aldosterone 10 and oxytocin 12 which is modulated by serotonin 14 and the pair of adrenaline 16 and cortisol 18 modulated by oxytocin 12. This relationship is shown in FIG. 2. It can be seen from the relationship how oxytocin 12 is both modulated and a modulator. As such the five neurotransmitters are interconnected and their interrelationship plays a role in the functioning of an individual's health processes.

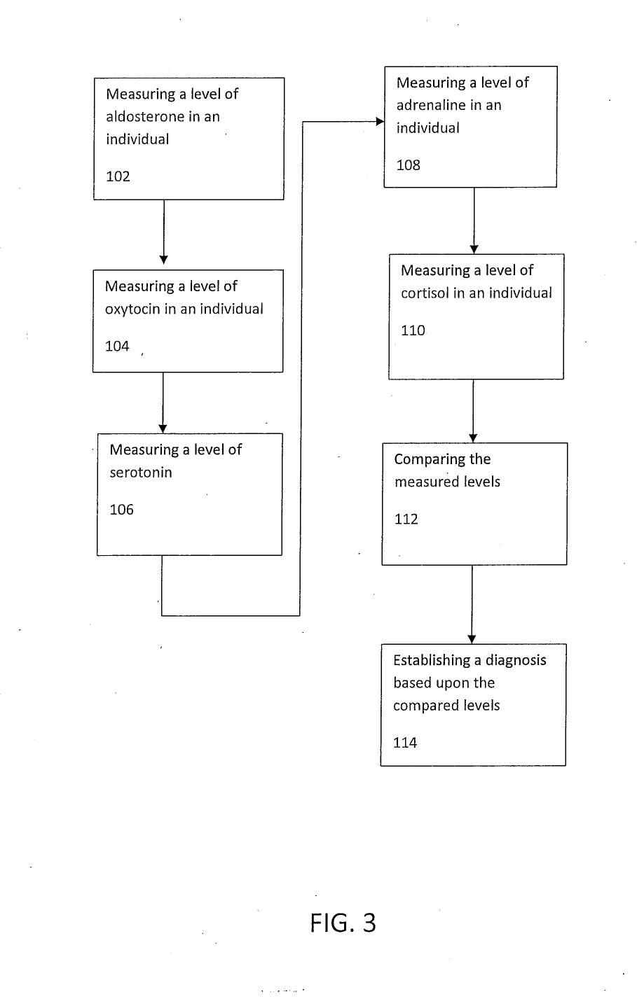

FIG. 3 shows the method of evaluating the homeostatic levels of two sets of modulated pairs in order to provide a base index for administering medication. In step 102 the level of aldosterone 10 is measured in an individual. The level of aldosterone 10 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat tears; saliva; mucus; phlegm; hair; fingernails; bone marrow. The measured level of aldosterone 10 in the body is expressed in various units depending upon the test employed in order to measure the level of the aldosterone 10. This level can be expressed by the variable X. There is a preferred range in which X may fall that varies depending upon the type of test that is used in order to measure the level of aldosterone 10. In an embodiment of the present invention a standard type of test is used, such as a blood test.

In step 104 the level of oxytocin 12 in an individual is measured. This level can be measured using one of the various processes listed above. The measured level of oxytocin 12 is preferably expressed in the same units of measurement as that used in the measurement of aldosterone 10 so as to more readily compare the levels with respect to each other in order to obtain a base level. This level can be expressed by the variable Y, There is a preferred range in which Y may fall that varies depending upon the type of test that is used in order to measure the level of oxytocin 12. In an embodiment of the present invention a standard type of test is used, such as a blood test.

In step 106 the level of serotonin 14 in an individual is measured. This level can be measured using one of the various processes listed above. The measured level of serotonin 14 is preferably expressed in the same units of measurement as that used in the measurement of aldosterone 10 so as to more readily compare the levels with respect to each other in order to obtain a base level. This level can be expressed by the variable Z. There is a preferred range in which Z may fall that varies depending upon the type of test that is used in order to measure the level of serotonin 14. In an embodiment of the present invention a standard type of test is used, such as a blood test.

In step 108, the level of adrenaline 16 in an individual is measured. This level can be measured using one of the various processes listed above. The measured level of adrenaline 16 is preferably expressed in the same units of measurement as that used in the measurement of aldosterone 10 so as to more readily compare the levels with respect to each other in order to obtain a base level. This level can be expressed by the variable A. There is a preferred range in which A may fall that varies depending upon the type of test that is used in order to measure the level of adrenaline 16. In an embodiment of the present invention a standard type of test is used, such as a blood test.

In step 110, the level of cortisol 18 in an individual is measured. This level can be measured using one of the various processes listed above. The measured level of cortisol 18 is preferably expressed in the same units of measurement as that used in the measurement of aldosterone 10 so as to more readily compare the levels with respect to each other in order to obtain a base level. This level can be expressed by the variable B. There is a preferred range in which B may fall that varies depending upon the type of test that is used in order to measure the level of cortisol 18. In an embodiment of the present invention a standard type of test is used, such as a blood test.

In step 112, the measured levels X, Y, Z, A and B are then compared to standard levels. This may be done in a number of different ways. One possible way in which this can be accomplished is to total the values of all of the levels (X+Y+Z+A+B) in order to establish sum level C. Another way of accomplishing this may be to determine the ratios of each of the substances to each other, e.g. X: Y, Y: Z, X: Z, Y: A, Y: B and A:B. The sum level C or the ratios can be used in step 114 in order to establish a diagnosis of the individual that can be used in determining the provision of medication and other matters related to the measured levels. The ratios or the sum level C are compared to predetermined standards in order to establish a diagnosis.

Using summation of the identified substances provides for a flexibility not found in traditional models, Whether or not one of the substances is too low or too high is immaterial provided the overall summation falls within normal ranges. The summation analysis reflects the interrelationship of the substances with each other. Using ratios of the individual substance with respect to each other also reveals the inter-relationship of the substances to each other and does not rely on the level of single substance in order to determine whether or not a person is within normal range.

An example of using the summation process is provided below using hypothetical numbers so as to make understanding of the process easier. Measured results may further be normalized to a set scale in order to make comparison and calculation easier provided all values are expressed in the same units. Actual numbers from a test such as a blood test are typically expressed in units of mmol/L.

Measuring aldosterone 10 via a blood test results in a number for X that results in a number 4, from within a range of 1-10. Measuring oxytocin 12 via a blood test results in a number for Y of 5 from within a range of 1-10. Measuring serotonin 14 via a blood test results in a number for Z of 6 from within a range of between 1-10. Measuring adrenaline 16 via a blood test results in a number for A of 4 from within a range of 1-10. Measuring cortisol 18 via a blood test results in a number for B of 6 from within a range of 1-10. These numbers are then totaled and results in a C value of 25. A value for C between 20 and 30 may indicate a normal level. If the sum level C is not normal then it may be used in order to administer one of the variable substances in order to place the sum level back into normal levels. Administration may be orally administered, e.g., pills, solutions, lozenges, etc.; intravenously administered solutions; modification of dietary supplements and/or other standard methods of delivery. After a period of time after administration, the levels of each of the substances may be retested in order to ascertain whether or not the administration was effective. Additionally, the levels may be measured in order to determine if more should be administered.

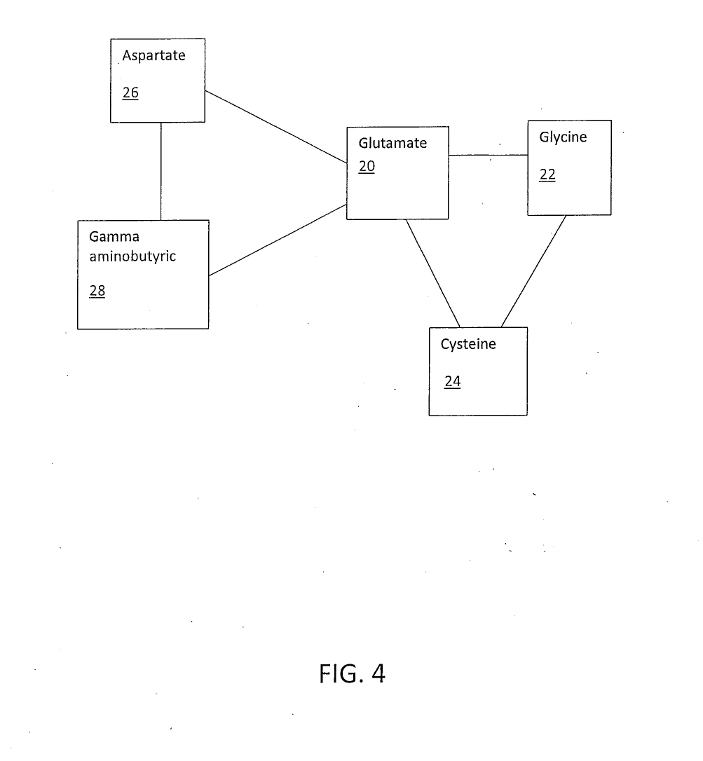

The second set is the pair of glutamate 20 and glycine 22 modulated by cysteine 24 and the pair of aspartate 26 and gamma amino butyric acid 28 modulated by glutamate 20. This relationship is shown in FIG. 4. It can be seen from the relationship how glutamate 20 is both modulated and a modulator. As such the five neurotransmitters are interconnected and their interrelationship plays a role in the functioning of an individual's health processes.

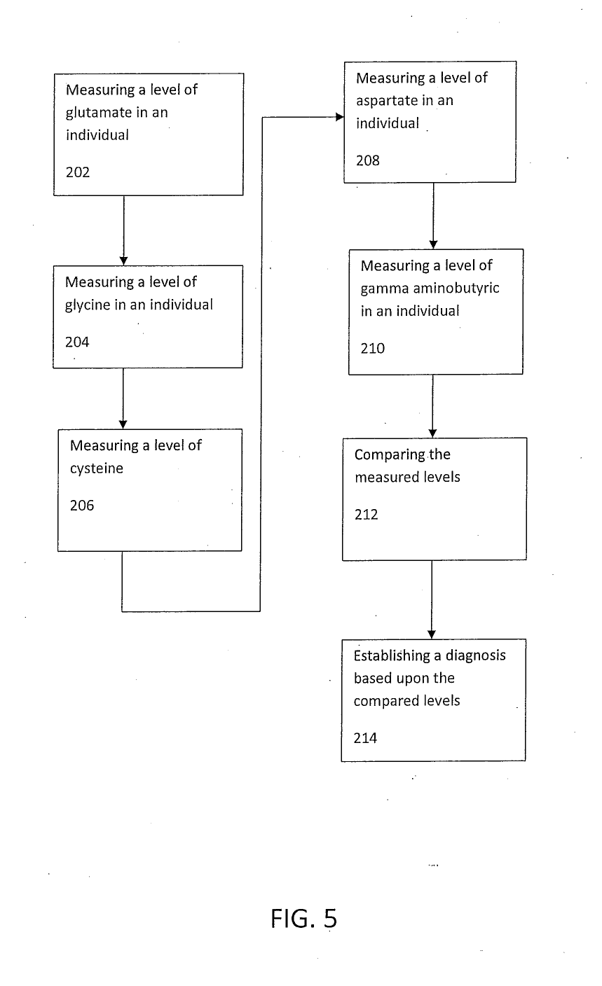

FIG. 5 shows the method of evaluating the homeostatic levels of two sets of modulated pairs in order to provide base index for administering medication. In step 202 the level of glutamate 20 is measured in an individual. The level of glutamate 20 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat tears; saliva; mucus; phlegm; hair; fingernails; bone marrow. The measured level of glutamate 20 in the body is expressed in various units depending upon the test employed in order to measure the level of the glutamate 20. This level can be expressed by the variable X. There is a preferred range in which X may fall that varies depending upon the type of test that is used in order to measure the level of glutamate 20. In an embodiment of the present invention a standard type of test is used, such as a blood test.

In step 204 the level of glycine 22 in an individual is measured. This level can be measured using one of the various processes listed above. The measured level of glycine 22 is preferably expressed in the same units of measurement as that used in the measurement of glutamate 20 so as to more readily compare the levels with respect to each other in order to obtain a base level. This level can be expressed by the variable Y. There is a preferred range in which Y may fall that vanes depending upon the type of test that is used in order to measure the level of glycine 22. In an embodiment of the present invention a standard type of test is used, such as a blood test.

In step 206 the level of cysteine 24 in an individual is measured. This level can be measured using one of the various processes listed above. The measured level of cysteine 24 is preferably expressed in the same units of measurement as that used in the measurement of glutamate 20 so as to more readily compare the levels with respect to each other in order to obtain a base level. This level can be expressed by the variable Z. There is a preferred range in which Z may fall that varies depending upon the type of test that is used in order to measure the level of cysteine 24. In an embodiment of the present invention a standard type of test is used, such as a blood test.

In step 208, the level of aspartate 26 in an individual is measured. This level can be measured using one of the various processes listed above. The measured level of aspartate 26 is preferably expressed in the same units of measurement as that used in the measurement of glutamate 20 so as to more readily compare the levels with respect to each other in order to obtain a base level. This level can be expressed by the variable A. There is a preferred range in which A may fall that varies depending upon the type of test that is used in order to measure the level of aspirate 26. In an embodiment of the present invention a standard type of test is used, such as a blood test.

In step 210, the level of gamma aminobutryic 28 in an individual is measured. This level can be measured using one of the various processes listed above. The measured level of gamma aminobutyric 28 is preferably expressed in the same units of measurement as that used in the measurement of glutamate 20 so as to more readily compare the levels with respect to each other in order to obtain a base level. This level can be expressed by the variable B. There is a preferred range in which B may fall that varies depending upon the type of test that is used in order to measure the level of gamma aminobutyric 28. In an embodiment of the present invention a standard type of test is used, such as a blood test.

In step 212, the measured levels X, Y, Z, A and B are then compared to standard levels. This may be done in a number of different ways. One possible way in which this can be accomplished is to total the values of all of the levels (X+Y+Z+A+B) in order to establish sum level C. Another way of accomplishing this may be to determine the ratios of each of the substances to each other, eg. X:Y, Y:Z, X:Z, X:A, X:B and A:B. The sum level C or the ratios can be used in step 214 in order to establish a diagnosis of the individual that can be used in determining the provision of medication and other matters related to the measured levels. The ratios or the sum level C are compared to predetermined standards in order to establish a diagnosis. The sum level C may also be used in order to administer one of the variable components in order to place the sum level back into normal levels. Administration may be orally administered, e.g., pills, solutions, lozenges, etc.; intravenously administered solutions; modification of dietary supplements and/or other standard methods of delivery. After a period of time after administration, the levels of each of the substances may be retested in order to ascertain whether or not the administration was effective. Additionally, the levels may be measured in order to determine if more should be administered.

An example of this is provided below using hypothetical numbers so as to make understanding of the process easier. Measured results may further be normalized to a set scale in order to make comparison and calculation easier provided the all values are expressed in the same units. Actual numbers from a test such as a blood test are typically expressed in units of mmol/L.

Measuring glutamate 20 via a blood test results in a number for X that results in a number 2, from within a range of 1-10. Measuring glycine 22 via a blood test results in a number for Y of 6 from within a range of 1-10. Measuring cysteine 24 via a blood test results in a number for Z of 6 from within a range of between 1-10. Measuring aspartate 26 via a blood test results in a number for A of 5 from within a range of 1-10. Measuring gamma aminobutyric 28 via a blood test results in a number for B of 7 from within a range of 1-10. These numbers are then totaled and results in a C value of 26. A value for C between 20 and 30 may indicate a normal level. Using summation of the identified substances provides for a flexibility not found in traditional models, Whether or not one of the substances is too low or too high is immaterial provided the overall summation falls within normal ranges. The summation analysis reflects the interrelationship of the substances with each other.

If the level was not normal one of the variable substances may be administered in order to place the sum level back into normal levels.

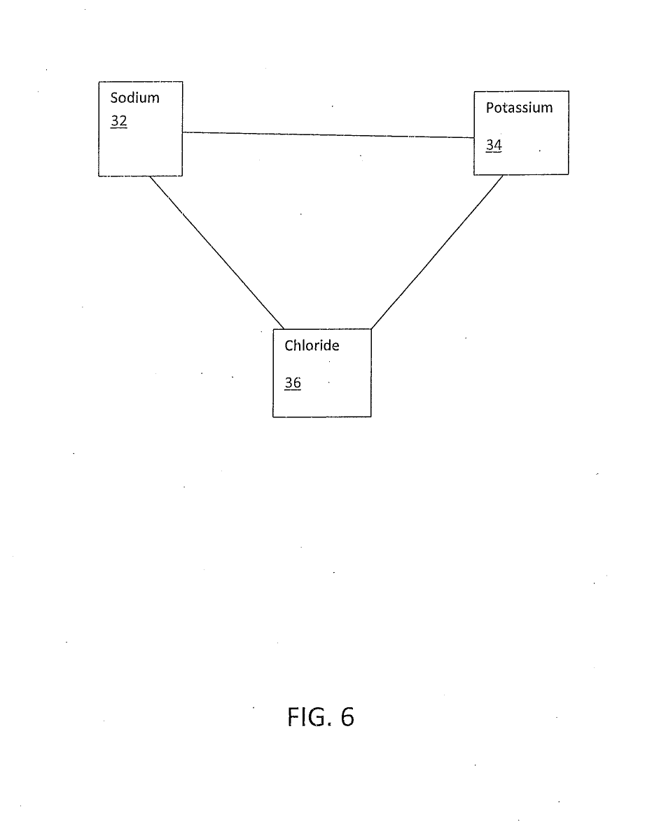

In FIG. 6, another example of a modulated pair is shown comprising sodium 32 and potassium 34 modulated by chloride 36. The pairs provided above also use the analysis method described below. The following steps are taken to establish the corollary data analysis methods to ascertain homeostatic levels.

The normal range and ratio of levels between sodium 32 and potassium 34 (a model example of a base pair) is determined using data currently available and accepted as being “normal”/“safe” levels for the individual substances that exist in diagnostic testing informatics in use today. The method of using similar data (levels that accepted as being “normal”/“safe” may serve as a standard for establishing base levels of substances mentioned above for the individual substances that exist in diagnostic testing informatics. High levels of one substance in relation to another in a base pair will lower the other substances level in the same test subject. In this example a low level of sodium 32 will result in a high level of potassium 34 and vice versa. Sodium 32 and potassium have a mutualistic, polarizing relationship Also in this example and in the body's effort to prevent levels of sodium 32 from being too low in order to maintain homeostasis in relation to potassium 34, chloride 36 functions as a modulator to boost the levels of sodium 32, thus bringing the pair, sodium 32 and potassium 34 back into balance.

Excessive chloride 36 in the body, caused by a disorder such as ingestion, will increase sodium 32 in the body. Too much chloride 36 will increase sodium 32 to excessive high levels, which in turn will have an effect that reduces potassium 34 to potentially excessive low levels. Identifying the level of chloride 36 will provide an indicator as to whether it is having an adverse effect on the base pair or if the problem is a result of excessive levels of sodium 32 or potassium 34 from some other source.

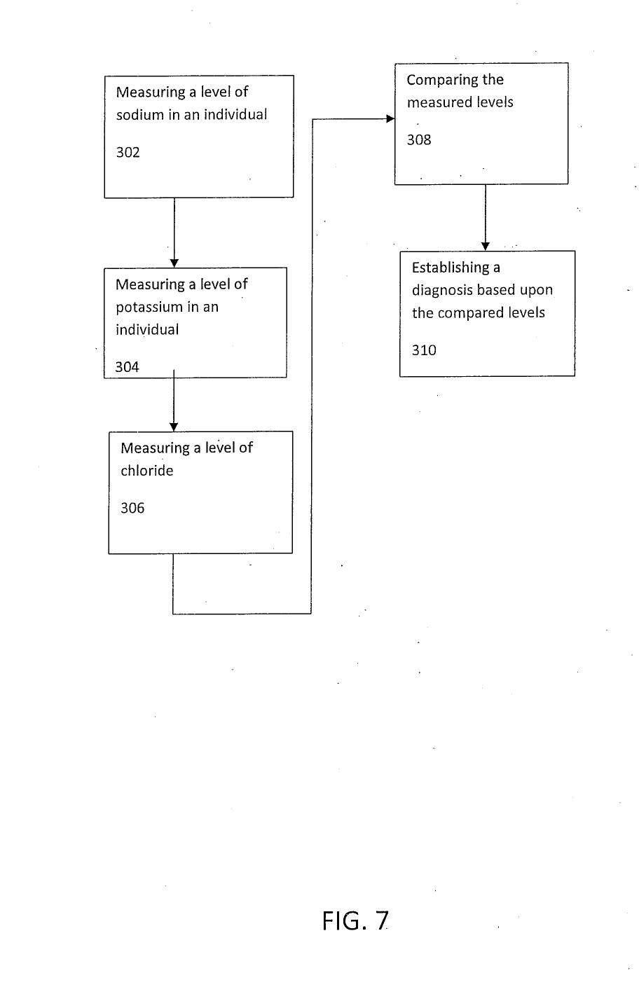

FIG. 7 shows the method of evaluating the homeostatic levels of a modulated pair in order to provide base index for administering medication. In step 302 the level of sodium 32 is measured in an individual. The level of sodium 32 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat tears; saliva; mucus; phlegm; hair; fingernails; bone marrow. The measured level of sodium 32 in the body is expressed in various units depending upon the test employed in order to measure the level of the glutamate 32. This level can be expressed by the variable X. There is a preferred range in which X may fall that varies depending upon the type of test that is used in order to measure the level of sodium 32. In an embodiment of the present invention a standard type of test is used, such as a blood test.

In step 304 the level of potassium 34 in an individual is measured. This level can be measured using one of the various processes listed above. The measured level of potassium 34 is preferably expressed in the same units of measurement as that used in the measurement of sodium 32 so as to more readily compare the levels with respect to each other in order to obtain a base level. This level can be expressed by the variable Y. There is a preferred range in which Y may fall that varies depending upon the type of test that is used in order to measure the level of potassium 32. In an embodiment of the present invention a standard type of test is used, such as a blood test.

In step 306 the level of chloride 36 in an individual is measured. This level can be measured using one of the various processes listed above. The measured level of chloride 36 is preferably expressed in the same units of measurement as that used in the measurement of sodium 32 so as to more readily compare the levels with respect to each other in order to obtain a base level. This level can be expressed by the variable Z. There is a preferred range in which Z may fall that varies depending upon the type of test that is used in order to measure the level of chloride 36. In an embodiment of the present invention a standard type of test is used, such as a blood test.

In step 308, the measured levels X, Y, Z, are then compared to standard levels. This may be done in a number of different ways. One possible way in which this can be accomplished is to total the values of all of the levels (X+Y+Z) in order to establish sum level C another way of accomplishing this may be determine the ratios of each of the substances to each other, e.g. X:Y, Y:Z and X:Z. This sum level C or the ratios can be used in step 310 in order to establish a diagnosis of the individual that can be used in determining the provision of medication and other matters related to the measured levels. The ratios or the sum level C are compared to predetermined standards in order to establish a diagnosis. The sum level C or the ratios may also be used in order to administer one of the variable components in order to place the sum level or ratios back into normal levels. Administration may be orally administered, e.g., pills, solutions, lozenges, etc.; intravenously administered solutions; modification of dietary supplements and/or other standard methods of delivery. After a period of time after administration, the levels of each of the substances may be retested in order to ascertain whether or not the administration was effective. Additionally, the levels may be measured in order to determine if more should be administered.

An example is now provided using ratios. The average currently accepted range of sodium 32 in blood is from between 135-148 mmol/L. The average currently accepted range of potassium 34 in blood is from between 3.5-5.5 mmol/L. The average currently accepted range of chloride in blood is from between 96-109 mmol/L. The midpoints of the average ranges are for Sodium 32, 140 mmol/L, for potassium 34, 4.35 mmol/L and for Chloride 36, 102.5 mmol/L.

The average ratio between sodium 32 and potassium 34 is 1:0.031 (for every 1 mmol/L of sodium 32 there is 0.031 mmol/L of potassium 34) or alternatively 32.18:1 (for every 32.18 mmol/L of sodium 32 there is 1 mmol/L of potassium 34). This ratio can then be used to further determine if modification to the one of the substances levels needs to be performed.

The average ratio between chloride 36 and sodium 32 is 1:1.36 (1 mmol/L chloride 36 to 1.36 mmol/L sodium 32) or C divided by A. Another ratio for the pair is 1:0.732 (1 mmol/L sodium 32 to 0.732 mmol/L of chloride 36). This ratio can then be used to further determine if modification to the one of the substances levels needs to be performed.

The average ratio between chloride and potassium is 1:0.042 (1 mmol/L chloride to 0.042 mmol/L potassium). Another ratio for the pair is 1:23.56 (1 mmol/L potassium to 23.56 mmol/L chloride). This ratio can then be used to further determine if modification to the one of the substances levels needs to be performed.

Once the values and ratios have been established, the detected levels of an individual test are compared to the established ratios to determine if abnormalities (results outside the normal, established values) exist. These abnormalities serve as indicators of imbalance and potential illness or disease (based on the modulated pair being tested) For example: Higher ratios of the chloride 36 to sodium 32 would indicate excessive chloride 36 can be contributing to higher levels of sodium 32 (e.g. 2:1.136) therefore lowering levels of potassium 34, providing a ratio that is lower than 1:0.031 for sodium 32 to potassium 34. Low levels of potassium 34 are biomarkers for spasms of skeletal muscles. The heart is comprised of striated muscles; a specialized form of skeletal muscle. Low levels of potassium 34 provide a biomarker for the increased probability of Cardiomyopathy. This process can be replicated for establishing levels and creating ratios for any substance referenced above provided acceptable data exists.

Correlating the data between the sodium 32, potassium 34 and chloride 36 provides patient care professionals with indicators to ascertain possible causes for potassium depletion and the ability to isolate which of the three substances, if any are causing problems. Based on low levels of potassium 34 and high levels of sodium 31, individually or in relation to chloride 36, patient care professionals can recommend dietary changes, prescribe medications in quantities specific to return sodium 32 and potassium 34 levels to these within the established range of homeostasis

In addition to the analysis methods provided above, various existing and yet to be developed diagnostic processes that measure outcomes involving these substances can be utilized. Examples of such methodologies include but are not limited to EEG, PET scans, use of MEG machines, SPECT analysis, functional and diffusion MRI technologies as well as other iterations thereof (fMRI and dMRI respectively), CT scans, and ultrasound.

The present invention provides the biological foundation that will enable the monitoring of relationships of modulated pairs of substances as they relate to medical care; including wellness, prevention and treatment pertaining to the mind and body.

It is to be understood, however, that even though numerous characteristics and advantages of the present invention have been set forth in the foregoing description, together with details of the invention, the disclosure is illustrative only, and changes may be made in detail to the full extent indicated by the broad general meaning of the terms in which the appended claims are expressed.

What is claimed is:

1. A method of determining a diagnosis comprising; measuring a level of a first substance in an individual; measuring a level of a second substance in an individual; measuring a level of a third substance in an individual; comparing the measured level of the first substance to the measured level of the second substance; comparing the measured level of the second substance to the measured level of the third substance and comparing the measured level of the third substance to the measured level of the first substance; and wherein the compared measured levels are further compared to predetermined levels to establish a diagnosis.

2. The method of claim 1, wherein the step of comparing comprises taking a first ratio of the measured level of the first substance to the second substance; taking a ratio of the second substance to the third substance and taking a ratio of the third substance to the first substance.

3. The method of claim 1, wherein the step of comparing comprises summing the measured level of the first substance, the measured level of the second substance and the measured level of the third substance.

4. The method of claim 1, wherein the first substance is sodium, the second substance is potassium, and the third substance is chloride.

5. The method of claim 1, wherein the first substance is selected from the group consisting of oxtocin, glutamate and chloride.

6. The method of claim 5, wherein the second substance is selected from the group consisting of sodium, cortisol and glycine.

7. The method of claim 6, wherein the third substance is selected from the group consisting of potassium, adrenaline and cysteine.

8. The method of claim 1, wherein measuring the level of the first substance comprises measuring the level of the first substance in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

9. The method of claim 1, wherein measuring the level of the second substance comprises measuring the level of the second substance in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

10. The method of claim 1, wherein measuring the level of first substance and measuring the level of the second substance comprises measuring the level of the first substance and the second substance in the same bodily excretion.

11. The method of claim 10, wherein the bodily excretion is blood.

12. The method of claim 1, further comprising administering the third substance based on the established diagnosis.

13. The method of claim 12, wherein the third substance is administered by provision of orally administered substances, intravenously administered solutions or modification of dietary supplements

14. The method of claim 1, wherein the first substance is aldosterone, the second substance is oxytocin, and the third substance is serotonin.

15. The method of claim 14, further comprising measuring the level of a fourth substance, wherein the fourth substance is adrenaline and further measuring the level of a fifth substance wherein the fifth substance is cortisol.

16. The method of claim 1, wherein the first substance is glutamate, the second substance is glycine, and the third substance is cysteine.

17. The method of claim 16, further comprising measuring the level of a fourth substance, wherein the fourth substance is aspartate and further measuring the level of a fifth substance wherein the fifth substance is gamma aminobutyric.

18. The method of claim 1, further comprising: administering the third substance based on the established diagnosis; re-measuring the level of the first substance in the individual after the step of administering; re-measuring measuring the level of the second substance in the individual after the step of administering; re-measuring the level of the third substance in the individual after the step of administering; and re-comparing the re-measured levels to the predetermined levels.

19. A method for establishing a diagnosis based upon compared levels of neurotransmitters: measuring a level of aldosterone in an individual; measuring a level of oxytocin in the individual; measuring a level of serotonin in the individual; measuring a level of adrenaline in the individual; measuring a level of cortisol in the individual; comparing the measured levels; and establishing a diagnosis based upon the compared measured levels.

20. A method for establishing a diagnosis based upon compared levels of neurotransmitters: measuring a level of glutamate in an individual; measuring a level of glycine in the individual; measuring a level of cysteine in the individual; measuring a level of aspartate in the individual; measuring a level of gamma aminobutyric in the individual; comparing the measured levels; and establishing a diagnosis based upon the compared measured levels.
